(19) United States
(12) Patent Application Publication
(10) Pub. No.: US 2015/0346193 A1
(43) Pub. Date: Dec. 3, 2015

(54) SYSTEM AND METHOD FOR LABELING ESSENTIAL OILS

(71) Applicant: Zija International, Inc., Lehi, UT (US)

(72) Inventor: Joshua James Plant, Provo, UT (US)

(73) Assignee: Zija International, Inc., Lehi, UT (US)

(21) Appl. No.: 14/459,065

(22) Filed: Aug. 13, 2014

Related U.S. Application Data
(60) Provisional application No. 62/005,889, filed on May 30, 2014.

(51) Int. Cl. G01N 33/52

(52) U.S. Cl. CPC G01N 33/523; G01N 2560/00; G01N 2458/15

(57) ABSTRACT

The disclosure extends to systems and methods of labeling compounds of essential oils with a marker. In an implementation, the labeling of the compounds of essential oils may comprise using a fluorescent dye that covalently links the hydroxyl group to the fluorescent dye, such that the compound may be detected using a fluorescence microscope.

### CROSS-REFERENCE TO RELATED APPLICATIONS

This application claims the benefit of U.S. Provisional Patent Application No. 62/005,889, filed on May 30, 2014, which is hereby incorporated by reference herein in its entirety, including but not limited to those portions that specifically appear hereinafter, the incorporation by reference being made with the following exception: In the event that any portion of the above-referenced application is inconsistent with this application, this application supersedes said above-referenced application.

### STATEMENT REGARDING FEDERALLY SPONSORED RESEARCH OR DEVELOPMENT

Not Applicable.

### BACKGROUND

The disclosure relates generally to essential oils, and more particularly, but not necessarily entirely, to systems and methods for labeling essential oils so that the presence of the labeled essential oil may be detected.

Essential oils have been used for centuries for their medicinal value. Essential oils are volatile liquids that are distilled from plants. Plants, shrubs, flowers, trees, roots, leaves, pedals, and seeds are all used in the distilling process to produce essential oils. Essential oils are believed to contain immune defense, oxygenating, regenerative properties that are inherent in all chlorophyll rich plants. Accordingly, essential oils are used to promote healthy living and lifestyles. Essential oils contain high amounts of oxygenating molecules that function to transport nutrients into cells where nutrients can be efficiently assimilated and utilized by the cells.

However, there has long been a mystique surrounding essential oils and their therapeutic abilities. Questions surrounding the efficacy often relate to whether essential oils are effectively entering into the cells of living organisms or whether the essential oils are merely processed by the body. Accordingly, there is a need to establish whether essential oils are entering into cells of living organisms. What is needed are systems and methods for labeling essential oils so that the essential oils may be detected to determine whether such oils are entering into the cells of living organisms. As will be seen, the disclosure provides such systems and methods for labeling essential oils in an effective and elegant manner.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1 illustrates an implementation of a method of labeling essential oils in accordance with the teachings and principles of the disclosure;

FIG. 2 illustrates an implementation of a method of labeling essential oils in accordance with the teachings and principles of the disclosure;

FIG. 3 illustrates an implementation of a method of labeling essential oils in accordance with the teachings and principles of the disclosure;

FIG. 4A illustrates a plurality of epithelial cells before being exposed to labeled essential oil in accordance with the teachings and principles of the disclosure; and

FIG. 4B illustrates a plurality of epithelial cells after being exposed to labeled essential oil and further illustrating the fluorescing of markers that may be covalently linked to one or more target compounds in the essential oil thereby establishing the uptake of the essential oil into the cells in accordance with the teachings and principles of the disclosure.

### DETAILED DESCRIPTION

The disclosure extends to systems and methods for labeling essential oils. In the following description of the disclosure, reference is made to the accompanying drawings, which form a part hereof, and in which is shown by way of illustration specific implementations in which the disclosure is may be practiced. It is understood that other implementations may be utilized and structural changes may be made without departing from the scope of the disclosure.

Before the systems, methods and processes for labeling essential oils are disclosed and described, it is to be understood that this disclosure is not limited to the particular embodiments, configurations, or process steps disclosed herein as such embodiments, configurations, or process steps may vary somewhat. It is also to be understood that the terminology employed herein is used for the purpose of describing particular embodiments only and is not intended to be limiting since the scope of the disclosure will be limited only by the appended claims, and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

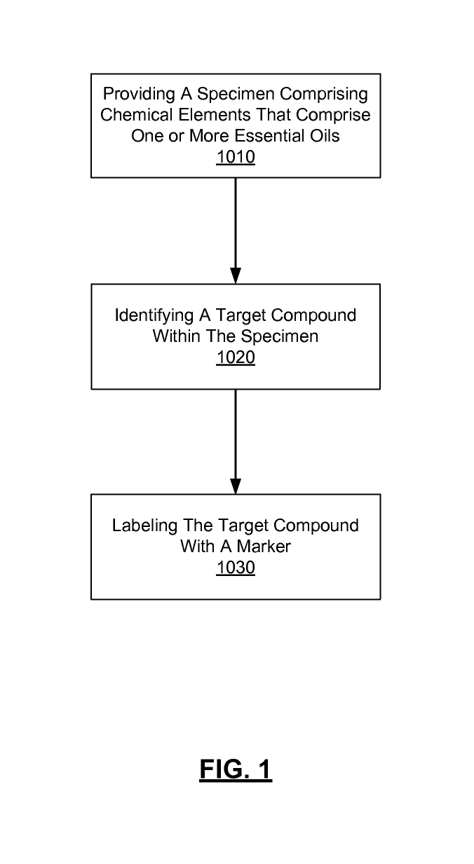

Referring now to FIG. 1, there is illustrated a system and method of labeling essential oils 1000. The system and method 1000 may comprise at 1010 providing a specimen having one or more chemical elements that comprise one or more essential oils. It will be appreciated that essential oils may include a concentrated hydrophobic liquid containing volatile aroma compounds found in natural sources, including plants. Essential oils may be referred to or may be known as volatile oils, ethereal oils, aetherolea, or oils of plants from which they were extracted. An oil may be considered essential in the sense that it contains the characteristic fragrance or properties of the plant from which it was obtained. It will be appreciated that essential oils do not form a distinctive category for any medical, pharmacological or culinary purpose and are not considered “essential” for health purposes.

Essential oils are generally extracted by distillation, often by using steam. Other processes include expression or solvent extraction. They are used in perfumes, cosmetics, soaps and other products, for flavoring food and drink, and for adding scents to incense and household cleaning products.

It will be appreciated that the disclosure contemplates the labeling of any and all essential oils. Essential oils may include, but are not necessarily limited to, Agar oil, Ajwain oil, Angelica root oil, Anise oil, Asafoetida, Balsam of Peru, Basil oil, Bay oil, Bergamot oil, Black Pepper, Buchu oil, Birch, Camphor, Cannabis flower essential oil, Caraway oil, Cardamom seed oil, Carrot seed oil, Cedarwood oil, Chamomile oil, Calamus Root, Cinnamon oil, Cistus species, Citronella oil, Clary Sage, Clove leaf oil, Coffee, Coriander, Costmary oil, Costus Root, Cranberry seed oil, Cubeb, Cumin oil/Black seed oil, Cypress, Cypriol, Curry leaf, Davana oil, Dill oil, Elecampane, Eucalyptus oil, Fennel seed oil, Fenugreek oil, Fir, Frankincense oil, Galangal, Galbanum, Geranium oil, Ginger oil, Goldenrod, Grapefruit oil, Henna oil, Helichrysum, Hickory nut oil, Horseradish oil, Hyssop, Idaho Tansy, Jasmine oil, Juniper berry oil, Laurus nobilis, Lavender oil, Ledum, Lemon oil, Lemongrass, Lime, Litsea cubeba oil, Linaloe, Mandarin, Marjoram, Melaleuca See Tea tree oil, Melissa oil (Lemon balm), Mentha arvensis oil/Mint oil, Mountain Savory, Mugwort oil, Mustard oil (essential oil), Myrrh oil, Myrtle, Neem oil or Neem Tree Oil, Neroli, Nutmeg, Orange oil, Oregano oil, Orris oil, Palo Santo, Parsley oil, Patchouli oil, Perilla essential oil, Pennyroyal oil, Peppermint oil, Petitgrain, Pine oil, Ravensara, Red Cedar, Roman Chamomile, Rose oil, Rosehip oil, Rosemary oil, Rosewood oil, Sage oil, spice star anise distilled to make star anise oil, Sandalwood oil, Sassafras oil, Savory oil, Schisandra oil, Spearmint oil, Spikenard, Spruce, Star anise oil, Tangerine, Tarragon oil, Tea tree oil, extracted from Melaleuca alternifolia, Thyme oil, Tsuga, Turmeric, Valerian, Vetiver oil (khus oil), Western red cedar, Wintergreen, Yarrow oil, Ylang-ylang and Zedoary.

The system and method 1000 may further comprise at 1020 identifying a target compound within the specimen. It will be appreciated that at 1030 the target compound may be labeled with a marker.

It will be appreciated that the target compound may be the molecule of interest in a chemical synthesis. The target compound may be, but is not necessarily limited to, a hydroxyl group, a sulfur group or sulfidyl group, any terpene, linalool, or eugenol that may be found in essential oils. For example, the target compound may be nerol, borneol, terpinen-4-ol, terpineol, geraniol, citronellol, menthol, or other molecules of interest that may be found in essential oils.

It is to be understood that terpenes are a large and diverse class of organic compounds produced by a variety of plants. For example, terpenes are produced in conifers, though terpenes may also be produced by some insects such as termites or swallowtail butterflies, which emit terpenes from their osmeteria. Terpenes may be strong-smelling, and thus may protect the plants that produce them by deterring parasites. Because of their aromatic properties, many terpenes are aromatic hydrocarbons and thus may have had a protective function. The difference between terpenes and terpenoids is that terpenes are hydrocarbons, whereas terpenoids contain additional functional groups.

In addition to their roles as end-products in many organisms, terpenes are major biosynthetic building blocks within nearly every living organism. When terpenes are modified chemically, such as by oxidation or rearrangement of the carbon skeleton, the resulting compounds may be generally referred to as terpenoids. It will be appreciated that the term terpene may include all terpenoids. It is to be understood that terpenoids may also be known as isoprenoids.

Terpenes and terpenoids are the primary constituents of the essential oils of many types of plants and flowers. Essential oils are used widely as natural flavor additives for food, as fragrances in perfumery, and in traditional and alternative medicines such as aromatherapy. Synthetic variations and derivatives of natural terpenes and terpenoids also greatly expand the variety of aromas used in perfumery and flavors used in food additives. For example, vitamin A is a terpene.

Terpenes may be released by trees more actively in warmer weather, acting as a natural form of cloud seeding. The clouds reflect sunlight, allowing the forest to regulate its temperature.

It will be appreciated that terpenes are derived biosynthetically from units of isoprene. Isoprene has the molecular formula C₅H₈. The basic molecular formulae of terpenes are multiples of (C₅H₈)ₙ where “n” is the number of linked isoprene units. The isoprene units may be linked together “head to tail” to form linear chains or they may be arranged to form rings. The isoprene unit may be considered as one of nature's common building blocks.

It will be understood that isoprene itself does not undergo the building process, but rather activated forms, isopentenyl pyrophosphate (IPP or also isopentenyl diphosphate) and dimethylallyl pyrophosphate (DMAPP or also dimethylallyl diphosphate), are the components in the biosynthetic pathway. IPP may be formed from acetyl-CoA via the intermediacy of mevalonic acid in the HMG-CoA reductase pathway. An alternative, unrelated biosynthesis pathway of IPP is known in some bacterial groups and the plastids of plants, the so-called MEP-pathway (2-Methyl-D-erythritol-4-phosphate), which is initiated from C5-sugars. In both biosynthesis pathways, IPP is isomerized to DMAPP by the enzyme isopentenyl pyrophosphate isomerase.

As chains of isoprene units are built up, the resulting terpenes are classified sequentially by size as hemiterpenes, monoterpenes, sesquiterpenes, diterpenes, sesterterpenes, triterpenes, sesquarterpenes, tetraterpenes, polyterpenes, and norisoprenoids, all of which are within the scope of the disclosure. Essentially, they are all synthesized by terpene synthase.

Linalool is a naturally occurring terpene alcohol chemical found in many flowers and spice plants with many commercial applications, the majority of which based on its pleasant scent (floral, with a touch of spiciness). It has other names such as β-linalool, linalyl alcohol, linaloyl oxide, p-linalool, allo-ocimenol, and 2,6-dimethyl-2,7-octadien-6-ol.

Linalool has a stereogenic center at C₃ and therefore there are two stereoisomers: (R)-(−)-linalool is also known as licareol and (S)-(+)-linalool is also known as coriandrol. Both enantiomeric forms are found in nature: (S)-linalool is found, for example, as a major constituent of the essential oils of coriander (Coriandrum sativum L. family Apiaceae) seed, palmarosa [Cymbopogon martinii var martinii (Roxb.) Wats., family Poaceae], and sweet orange (Citrus sinensis Osbeck, family Rutaceae) flowers. (R)-linalool is present in lavender (Lavandula officinalis Chaix, family Lamiaceae), bay laurel (Laurus nobilis, family Lauraceae), and sweet basil (Ocimum basilicum, family Lamiaceae), among others.

Each enantiomer evokes different neural responses in humans, and therefore are classified as possessing distinct scents. (S)-(+)-Linalool is perceived as sweet, floral, petitgrain-like (odor threshold 7.4 ppb) and the (R)-form as more woody and lavender-like (odor threshold 0.8 ppb).

In higher plants linalool, as other monoterpenoids, is produced from isopentenyl pyrophosphate via the universal isoprenoid intermediate geranyl pyrophosphate, through a class of membrane-bound enzymes named monoterpene synthases. One of these, linalool synthase (LIS), has been reported to produce (S)-linalool in several floral tissues.

Eugenol is a phenylpropene, an allyl chain-substituted guaiacol. Eugenol is a member of the phenylpropanoids class of chemical compounds. It is a clear to pale yellow oily liquid extracted from certain essential oils especially from clove oil, nutmeg, cinnamon, basil and bay leaf, among others. It is present in various concentrations. Eugenol may be used in perfumeries, flavorings, essential oils and in medicine as a local antiseptic and anesthetic.

Eugenol may be present in various plants, including, but not necessarily limited to, cloves (syzygium aromaticum), wormwood, cinnamon, cinnamomum tamala, nutmeg (myristica fragrans), ocimum basilicum—sweet basil, ocimum gratissimum, African basil, ocimum tenuiflorum (ocimum sanctum)—Tulsi or Holy Basil, Japanese star anise, lemon balm, dill, pimenta racemosa, vanilla, bay laurel, and celery.

It will be appreciated that the marker may be a labeling molecule or a molecule used to label the target compound. For example, the marker may be any fluorescent marker or fluorescent dye. It will be appreciated that the fluorescent marker or dye may comprise a rhodamine, such as sulforhodamine 101, or an azo compound, such as an azo dye.

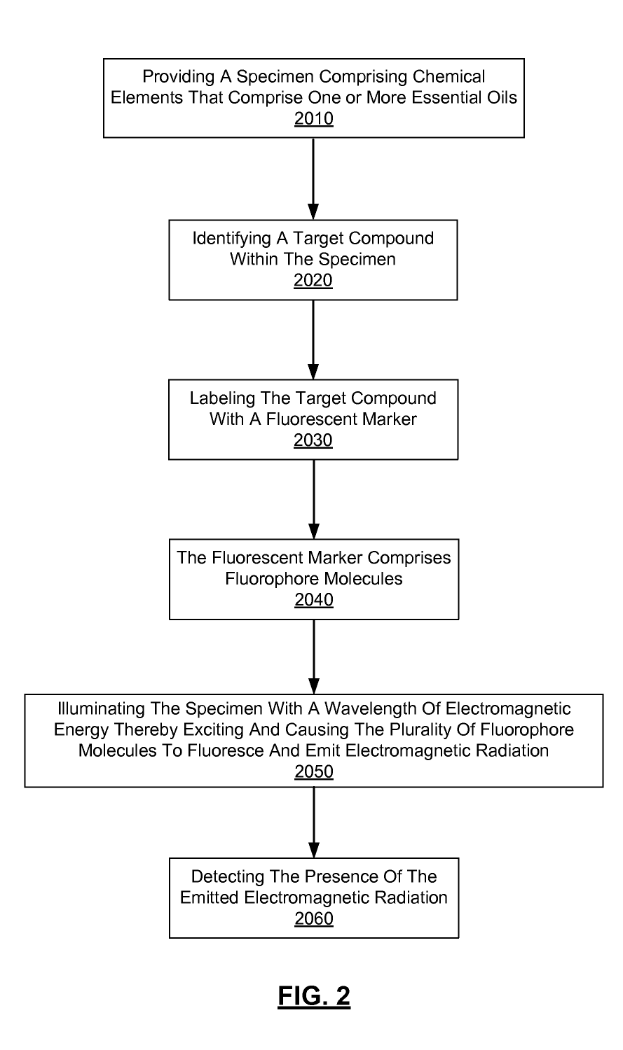

Referring now to FIG. 2, it will be appreciated that the disclosure relating to FIG. 1 is hereby incorporated into the description of FIG. 2. The system and method of labeling an essential oil 2000 illustrated in FIG. 2 in accordance with the disclosure may comprise: providing a specimen having one or more chemical elements that comprise one or more essential oils at 2010. The system and method 2000 may further comprise at 2020 identifying a target compound within the specimen. It will be appreciated that at 2030 the target compound may be labeled with a marker, such as a fluorescent marker. It will be appreciated that the fluorescent marker may comprise a plurality of electrophilic fluorophore molecules as illustrated at 2040 and the system and method 2000 may thus further comprise fluorescently labeling the target compound with a plurality of electrophilic fluorophore molecules. At 2050, the system and method 2000 may comprise illuminating the specimen with a wavelength of electromagnetic energy or radiation thereby exciting the plurality of fluorophore molecules causing them to fluoresce and emit electromagnetic energy or radiation of a different wavelength than the illumination wavelength. At 2060, the system and method 2000 may comprise detecting the presence of the emitted electromagnetic energy or radiation.

When the specimen is illuminated with electromagnetic energy or radiation of a specific wavelength (or plurality of wavelengths), the electromagnetic energy or radiation is absorbed by the fluorophore molecules, thereby causing those molecules to emit light of a different, longer wavelength, which may be a different wavelength and color than the absorbed electromagnetic energy or radiation.

The illuminated electromagnetic energy or radiation may be separated from the much weaker emitted fluorescence through the use of a spectral emission filter or other filter. Typical components of a fluorescence microscope may include: a light source, wherein the light source may comprise a xenon arc lamp or mercury-vapor lamp, high-power LEDs, or lasers; an excitation filter; a dichroic mirror (or dichroic beam splitter); and an emission filter. It will be appreciated that the filters and the dichroic may be chosen to match the spectral excitation and emission characteristics of the fluorophore molecules used to label the specimen. In this manner, the distribution of a single fluorophore (color) is imaged at a time. Multi-color images of several types of fluorophores may be composed by combining or using a plurality of single-color images, or using a plurality of types of fluorophore molecules to create a multi-color image that is composed by combining several single-color images.

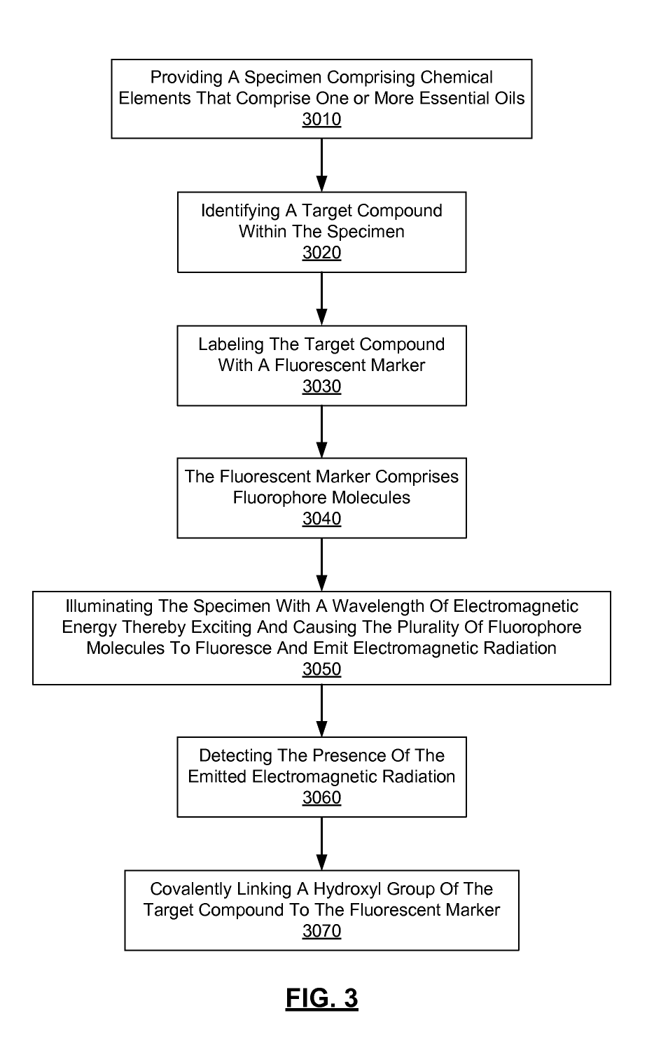

Referring now to FIG. 3, it will be appreciated that the disclosure relating to FIGS. 1 and 2 are hereby incorporated into the description of FIG. 3. The system and method of labeling an essential oil 3000 illustrated in FIG. 3 in accordance with the disclosure may comprise: providing a specimen having one or more chemical elements that comprise one or more essential oils at 3010. The system and method 3000 may further comprise at 3020 identifying a target compound within the specimen. It will be appreciated that at 3030 the target compound may be labeled with a marker, such as a fluorescent marker. It will be appreciated that the fluorescent marker may comprise a plurality of electrophilic fluorophore molecules as illustrated at 3040 and the system and method 3000 may thus further comprise fluorescently labeling the target compound with a plurality of electrophilic fluorophore molecules. At 3050, the system and method 3000 may comprise illuminating the specimen with a wavelength of electromagnetic energy or radiation thereby exciting the plurality of fluorophore molecules causing them to fluoresce and emit electromagnetic energy or radiation of a different wavelength than the illumination wavelength. At 3060, the system and method 3000 may comprise detecting the presence of the emitted electromagnetic energy or radiation. At 3070, the system and method 3000 may comprise covalently linking a hydroxyl group of the target compound to the marker, such as a fluorescent or other marker. The system and method 3000 may further comprise covalently linking a hydroxyl group of the target compound with an electrophilic fluorophore molecule.

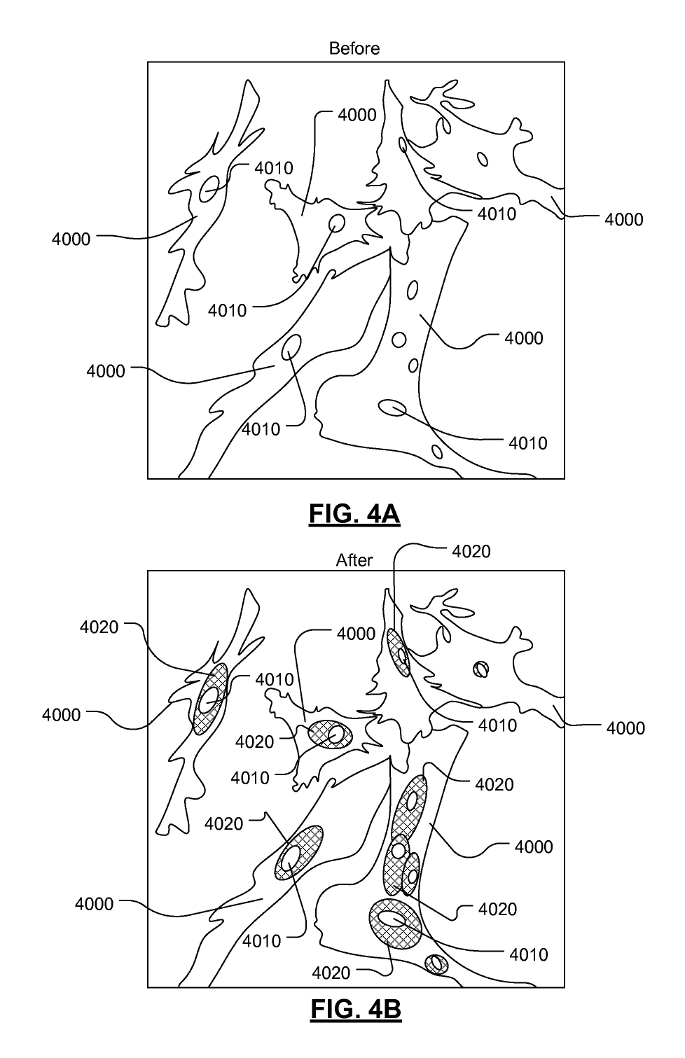

Referring now to FIGS. 4A and 4B, it will be appreciated that the disclosure relating to FIGS. 1-3 are hereby incorporated into the description of FIGS. 4A and 4B. It will be appreciated that FIG. 4A illustrates a plurality of epithelial cells before being exposed to labeled essential oil in accordance with the teachings and principles of the disclosure. Whereas, FIG. 4B illustrates a plurality of epithelial cells after being exposed to labeled essential oil and further illustrates the fluorescing of markers that may be covalently linked to one or more target compounds in the essential oil thereby establishing the uptake of the essential oil into the cells in accordance with the teachings and principles of the disclosure.

In FIGS. 4A and 4B there is illustrated a before and after illustration of a plurality of epithelial cells 4000 with nuclei 4010. It will be appreciated that FIG. 4A illustrates the epithelial cells before the addition of an essential oil, which has been labeled with a marker, is introduced to the cell. FIG. 4B illustrates the epithelial cells after the addition of an essential oil, which has been labeled with a marker, is introduced to the cell. It will be appreciated that the target compound of interest in the essential oil may be marked with any of the markers noted herein without departing from the scope of the disclosure.

Fluorescent labeling of one or more target compounds in an essential oil may be used for detecting and tracking the presence of a protein or other labeled molecule via a fluorescence microscope, flow cytometer or some other fluorescence reading instrument. A fluorescently labeled molecule may be detected, tracked, and/or traced as it is taken-up by and enters into living cells, thereby confirming the presence of the labeled molecule within living cells. Fluorescently labeled molecules can be useful in localization of a target within a cell, flow cytometry (FACS) analysis, western blot assays, and other immune-analytical methods. Referring specifically to FIG. 4B, it will be appreciated that the essential oil 4020, which has been labeled with a fluorescent marker, and has been taken-up or utilized by the epithelial cells 4000. The essential oil 4020 is illustrated as it is being concentrated around or otherwise surrounding the nuclei 4010 of the epithelial cells. It will be appreciated and understood that the illustrations shown in FIGS. 4A and 4B are illustrative and exemplary of any essential oil, which has been marked with a marker, that may be utilized by any living cell. All combinations of essential oils and markers so that the visualization, tracking, and/or detection of the essential oil into or within the cell are intended to fall within the scope of the disclosure.

The foregoing description has been presented for the purposes of illustration and description. It is not intended to be exhaustive or to limit the disclosure to the precise form or implementations disclosed. It will be appreciated that many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A method of labeling essential oils comprising:
providing a specimen comprising chemical elements that comprise one or more essential oils;
identifying a target compound within the specimen; and
labeling the target compound with a marker.

2. The method of claim 1, wherein the method further comprises fluorescently labeling the target compound with a plurality of electrophilic fluorophore molecules.

3. The method of claim 2, wherein the method further comprises illuminating the specimen with a wavelength of electromagnetic energy thereby exciting and causing the electrophilic fluorophore molecules to fluoresce and emit electromagnetic radiation.

4. The method of claim 3, wherein the method further comprises detecting the presence of the emitted electromagnetic radiation.

5. The method of claim 1, wherein the method further comprises covalently linking a hydroxyl group of the target compound to the marker.

6. The method of claim 2, covalently linking a hydroxyl group of the target compound with an electrophilic fluorophore molecule.

7. The method of claim 1, wherein the marker is a fluorescent dye.

8. The method of claim 7, wherein the fluorescent dye comprises a rhodamine.

9. The method of claim 8, wherein the rhodamine is sulforhodamine 101.

10. The method of claim 7, wherein the fluorescent dye comprises an azo compound.

11. The method of claim 10, wherein the azo compound is an azo dye.

12. The method of claim 7, wherein the fluorescent dye is detected using a fluorescence microscope.

13. The method of claim 3, wherein the fluorophore molecules absorb a specific wavelength of electromagnetic energy and emit electromagnetic energy that is different than the absorbed electromagnetic energy.

14. The method of claim 13, wherein the illumination light is separated from the emitted fluorescence by a spectral emission filter.

15. The method of claim 14, wherein the spectral emission filter matches the spectral excitation and emission characteristics of the fluorophore molecules used to label the specimen.

16. The method of claim 1, wherein the method further comprises using a plurality of types of fluorophore molecules to create a multi-color image that is composed by combining several single-color images.

17. The method of claim 1, wherein the target compound is a terpene.

18. The method of claim 1, wherein the target compound is a hydroxy-terpene.

19. The method of claim 1, wherein the target compound is linalool.

20. The method of claim 1, wherein the target compound is eugenol.

21. The method of claim 1, wherein the target compound comprises a hydroxyl group.

22. The method of claim 1, wherein the target compound comprises a sulfur group.

23. A method of labeling essential oils comprising:
providing a specimen comprising chemical elements that comprise one or more essential oils;
identifying a target compound within the specimen;
labeling the target compound with a fluorescent marker, wherein the fluorescent marker comprises a plurality of electrophilic fluorophore molecules;
illuminating the specimen with a wavelength of electromagnetic energy thereby exciting and causing the plurality of electrophilic fluorophore molecules to fluoresce and emit electromagnetic radiation; and
detecting the presence of the emitted electromagnetic radiation.

24. The method of claim 23, wherein the method further comprises covalently linking a hydroxyl group of the target compound to the fluorescent marker.

25. The method of claim 24, covalently linking a hydroxyl group of the target compound with a corresponding electrophilic fluorophore molecule.

26. The method of claim 23, wherein the marker is a fluorescent dye.

27. The method of claim 26, wherein the fluorescent dye comprises a rhodamine.

28. The method of claim 27, wherein the rhodamine is sulforhodamine 101.

29. The method of claim 26, wherein the fluorescent dye comprises an azo compound.

30. The method of claim 29, wherein the azo compound is an azo dye.

31. The method of claim 26, wherein the fluorescent dye is detected using a fluorescence microscope.

32. The method of claim 23, wherein the fluorophore molecules absorb a specific wavelength of electromagnetic energy and emit electromagnetic energy that is different than the absorbed electromagnetic energy.

33. The method of claim 32, wherein the illumination light is separated from the emitted fluorescence by a spectral emission filter.

34. The method of claim 33, wherein the spectral emission filter matches the spectral excitation and emission characteristics of the fluorophore molecules used to label the specimen.

35. The method of claim 23, wherein the method further comprises using a plurality of types of fluorophore molecules to create a multi-color image that is composed by combining several single-color images.

36. The method of claim 23, wherein the target compound is a terpene.

37. The method of claim 23, wherein the target compound is a hydroxy-terpene.

38. The method of claim 23, wherein the target compound is linalool.

39. The method of claim 23, wherein the target compound is eugenol.

40. The method of claim 23, wherein the target compound comprises a hydroxyl group.

41. The method of claim 23, wherein the target compound comprises a sulfur group.
